(19) United States
(12) Patent Application Publication
(10) Pub. No.: US 2024/0006016 A1
(43) Pub. Date: Jan. 4, 2024

(54) MACHINE LEARNING ENABLED METHODS FOR OPTIMAL INFERENCE AND DESIGN OF EXPERIMENTS FOR MECHANISTIC BIOLOGICAL MODELS

(71) Applicant: The Regents of the University of California, Oakland, CA (US)

(72) Inventor: Vincent Zaballa, Irvine, CA (US)

(21) Appl. No.: 18/217,513

(22) Filed: Jun. 30, 2023

Related U.S. Application Data
(60) Provisional application No. 63/357,625, filed on Jun. 30, 2022.

(51) Int. Cl. G16B 5/00; G16B 15/30; G16B 40/20

(52) U.S. Cl. CPC G16B 5/00; G16B 15/30; G16B 40/20

(57) ABSTRACT

This disclosure provides methods for optimal inference and design of experiments for mechanistic biological models to identify and/or rank compounds or agents that modulate a targeted cellular biological process to a statistically significant degree.

### CROSS REFERENCE TO RELATED APPLICATIONS

This application claims priority under 35 U.S.C. § 119 from Provisional Application Ser. No. 63/357,625, filed Jun. 30, 2022, the disclosure of which is incorporated herein by reference.

### STATEMENT OF GOVERNMENT SUPPORT

This invention was made with government support under Grant Nos. R01GM134418 and F31GM145188 awarded by the National Institutes of Health. The government has certain rights in the invention.

### TECHNICAL FIELD

This disclosure provides methods for optimal inference and design of experiments for mechanistic biological models to identify and/or rank compounds or agents that modulate a targeted cellular biological process to a statistically significant degree.

### BACKGROUND

Biological signaling pathways based upon proteins binding to one another to relay a signal for genetic expression, such as the Bone Morphogenetic Protein (BMP) signaling pathway, can be modeled by mass action kinetics and conservation laws that result in non-closed form polynomial equations. Accurately determining parameters of biological pathways that represent physically relevant features, such as binding affinity of proteins and their associated uncertainty, presents a challenge for biological models lacking an explicit likelihood function. Additionally, parameterizing non-closed form biological models requires copious amounts of data from expensive perturbation-response experiments to fit model parameters.

### SUMMARY

Many drugs fail because they are designed from a top-down perspective that ignores important biology. In direct contrast, the disclosure provides methods and methodology that expand the understanding of systems biology by characterizing the operation of the targeted biological system, and design drugs to intervene when the targeted biological system is not functioning correctly, e.g., in the case of diseases. In particular the methods disclosed herein utilize an innovative algorithm for system biology applications, including dosing cells with optimal hypotheses; building a machine-generated model using the data; and improving the machine-generate model using machine learning protocols with more data; and designing drugs to intervene in accurate models of cell biology.

Systems biology seeks to create math models of biological systems to reduce inherent biological complexity and provide predictions for applications such as therapeutic development. However, it remains a challenge to determine which math model is correct and how to arrive optimally at the answer. The methods of the disclosure utilize an algorithm for automated biological model selection using mathematical models of systems biology and likelihood free inference machine learning methods. Methods utilizing the algorithm showed improved performance in arriving at correct models without a priori information over conventional heuristics used in experimental biology and random search. This method shows promise to accelerate biological basic science and drug discovery.

A method that utilizes computer-implemented models and data from biological experiments in a machine learning model to identify and/or rank small molecule drug(s) and/or biologic(s) that modulate a targeted cellular biological process to a statistically significant degree, the process comprising: (A) obtaining cells from a subject or generating recombinant cells that elicit a measurable or trackable cellular functional response to small molecule drug(s) and/or biologic(s) on a targeted biological process; (B) training a first machine learning model with a plurality of computer-implemented models that model the targeted biological process, and which define prior probabilities in the models' parameters and models' marginal likelihood; (C) training a second machine learning model to estimate the mutual information between observed data and computer-implemented models' parameters, to design experiments to optimally perturb the modeled biological process with the small molecule(s) and/or biologic(s); (D) performing biological experiments with the cells from step (A) with small molecule drug(s) and/or biologic(s) identified from step (C) to generate measurable or observable cellular functional response data, the biological experiments being designed from the plurality of computer-implemented models' prior probabilities and binding affinity of the small molecule drug(s) and/or biologic(s) to a biological component of the targeted biological process; E) retraining the second machine learning model of step (C) using the measured or observed cellular functional response data to update: (i) the binding affinities of the targeted biological pathway, (ii) the small molecule drug(s) and/or biologic(s) binding affinity to the biological component, and (iii) to indicate which model of the plurality of computer-implemented models most accurately models the targeted biological process; (F) repeating steps (C) to (E) until small molecule drug(s) and/or biologic(s) are identified that perturb the targeted biological process until a Z-factor of 0.5 to 1.0 is determined, wherein if a plurality of small molecule drug(s) and/or biologic(s) are identified then the process ranks the small molecule drug(s) and/or biologic(s) by their activity in perturbing the targeted biological process. In another embodiment, the recombinant cells comprise a reporter gene or marker that is used to measure or track the cellular functional response to small molecule drug(s) and/or biologic(s) on a targeted biological process. In yet another embodiment, the cellular functional response to small molecule drug(s) and/or biologic(s) on a targeted biological process can be measured or tracked using luminescence, fluorescence or chemiluminescence produced by the reporter gene or marker. In a further embodiment, the cellular functional response to small molecule drug(s) and/or biologic(s) on a targeted biological process can be measured or tracked based upon changes in gene expression. In yet a further embodiment, gene expression can be measured or tracked using microarrays, sequencing, immunoassays, or biochips. In a certain embodiment, the cells obtained from a subject or the recombinant cells, are associated with a disease or disorder. In another embodiment, the disease or disorder is selected from an infectious disease, a deficiency disease, a genetic hereditary disease, a non-genetic hereditary disease, a physiological disease, an idiopathic disease, and a neoplastic disease. In another embodiment, one or more of the biological experiments are performed using high throughput screening with small molecule drugs and/or biologics from compound libraries. In a further embodiment, the biologic(s) are proteins or peptides. In yet a further embodiment, the plurality of computer-implemented models are mathematical models and/or models that predict protein structures when complexed with small molecule drugs and/or biologics. In a certain embodiment, the targeted biological process is a targeted biological signaling pathway. In another embodiment, the targeted biological signaling pathway is associated with a disease or disorder. In a further embodiment, the small molecule drugs and/or biologics modulate the activity of a biological component of the targeted biological signaling pathway. In yet a further embodiment, the targeted biological signaling pathway regulates growth, metabolism, or interactions and communications between cells. In another embodiment, the parameters of the plurality of computer-implemented models have user defined prior probabilities and marginal likelihoods.

In a particular embodiment, the disclosure also provides a method that utilizes computer-implemented models and data from biological experiments in a machine learning model to identify and/or perturbagen(s) that modulate a biological pathway to a statistically significant degree, the process comprising: (1) predicting the effect of perturbagen(s) on a biological pathway in a cellular system by using a plurality of different computer-generated models, wherein each computer-generated model provides a probable result as to the effect of perturbagen(s) on the biological pathway; (2) providing cells or a cellular system that elicits a measurable or trackable cellular functional response to perturbagen(s); (3) contacting the cells or cellular system with varying concentrations and/or combinations of perturbagens to modulate the activity of the biological pathway, and capturing phenotypic data resulting therefrom; (4) training a first machine learning model with the phenotypic data to infer the uncertainty distribution of parameters of the plurality of computer-generated models, and the probable results of each computer-generated model; (5) using the uncertainty distribution of parameters of the plurality of computer-generated models and the probability from each biological model to design additional sets of biological experiments in step (3), wherein steps (3)-(5) are repeated until perturbagen(s) are identified that perturb the biological pathway with a Z-factor from 0.5 to 1.0; and ((6) optionally, designing additional small molecule drugs and/or protein biologics based upon chemically modifying the perturbagen(s) identified in step (5).

In a further embodiment, the plurality of different computer-implemented models are mathematical models and/or models that predict protein structures when complexed with perturbagen(s). In yet a further embodiment the cellular functional response to perturbagen(s) on biological pathway can be measured or tracked using luminescence, fluorescence or chemiluminescence produced by a reporter gene or marker, or by measuring changes in gene expression. In another embodiment, the cells or cellular system are contacted with varying concentrations and/or combinations of perturbagens using a high through screening assay.

In a particular embodiment, the disclosure also provides the methods disclosed herein can also employ an algorithm as substantially described or diagramed herein.

### DESCRIPTION OF DRAWINGS

FIG. 1 displays the code for the Simulation-Based Inference Design Of Experiment for Biological Mechanistic Acyclic Networks (SBIDOEMAN) algorithm that can be implemented using machine learning protocols.

FIG. 2 presents a comparison of the search policy of the SBIDOEMAN and random search across an ensemble of models shows an improvement in the convergence of the SBIDOEMAN to the true value with less variance for both onestep (top) and twostep (bottom) models. For the onestep model, a simpler model with only two unknown parameters, SBIDOEMAN arrives at an accurate MAP estimate of the true parameter values with RMSE of 0.01±0.03 in just 2 designs. When examining the difference between experimental design policies in the twostep model, which has 3 unknown parameters, SBIDOEMAN showed more gradual improvement over random search to arrive at a lower RMSE MAP estimate of the correct held-out parameter values. However, improvement can qualitatively be seen by the last design, indicating that more designs may be required for more complex models to converge but that SBIDOEMAN is more efficient at arriving at true parameter values than random search.

FIG. 3 provides a comparison of different normalizing flows by ensembles of SBIDOEMAN. As shown, the MDN architectures (top) had increased variance in RMSE values over experimental runs while the NSF architecture (bottom) converged more rapidly and with less variance. The color of the lines indicates the ranking of the final RMSE, where red represents the highest RMSE and blue represents the lowest final RMSE.

FIG. 4 presents a Bayesian Model Averaging the algorithm SBIDOEMAN. For the choice of hyperparameters, NM=5000, NA=5, NS=1000, NR=5, NE=5, a SNLE q_{ϕ}(x|θ) density estimator, starting box uniform priors for p(θ), and uniform priors for p( [symbol] ᵢ) were used. Fifty simulations at a time limit of 10 hours were evaluated. For the one-step model, the random choice had 14 simulations finish, equidistant had 26 simulations finish, and SBIDOEMAN BMA had 15 simulations finish. For the two-step model, random choice had 21 simulations finish, equidistant had 25 finish, and SBIDOEMAN BMA had 16 finish.

FIG. 5 shows final Bayes Factor (BF) after 5 design rounds and an ensemble of models. Compared to controls for both models, SBIDOEMAN BMA performed an order of magnitude better on the one-step model and performed more than two times better than control policies of the two-step model.

FIG. 6 shows the change in Bayes Factor (BF), p(twostep)/p(onestep), over design round when the one-step (top) and two-step (bottom) models are true. The strong evidence threshold for both models is labeled in lighter gray. Top: When the one-step model is true, SBIDOEMAN BF model trends down, indicating the one-step model is true and outperforms random search by the final design. The median BF value for the SBIDOEMAN model strongly suggests the one-step model is true by the fifth round. Bottom: When the two-step model is true the median value of the SBIDOEMAN BF trends upwards, indicating the two-step model is true, and has a median trend that outperforms the competing random search by the last three designs. The two-step model's final value indicates only moderate evidence in favor of the true two-step model.

FIG. 7 demonstrates Bayesian Model Averaging for the SBIDOEMAN algorithm (termed herein as SBIDOEMAN BMA).

FIG. 8 demonstrates that biophysical information can be used to improve structure prediction or pathway parameters with the SBIDOEMAN BMA algorithm.

FIG. 9A-B provides (A) representation of the final result of successful inhibition of protein-protein interactions using the SBIDOEMAN algorithm. Protein interaction is inhibited by a novel therapeutic, small molecule or biologic, that successfully inhibits protein binding in the pathway. (B) Schematic of the optimization process for inhibiting protein interaction in, e.g., the BMP pathway. A therapeutic is designed to inhibit the pathway given the known data about the pathway's parameter. After gathering experimental data, knowledge about the pathway, structure of the proteins in the pathway, and which therapeutic is updated.

FIG. 10A-B presents an (A) exemplary user interface for identifying which biological model may underlie the true biological process, and how experiments are designed for that hypothesis. (B) Exemplary user interface for design of a drug for a given pathway of interest.

FIG. 11 presents a flowchart of an exemplary process that utilizes computer-implemented models and data from biological experiments in a machine learning model to identify and/or rank small molecule drug(s) and/or biologic(s) that modulate a cellular biological process.

### DETAILED DESCRIPTION

As used herein and in the appended claims, the singular forms “a,” “an,” and “the” include plural referents unless the context clearly dictates otherwise. Thus, for example, reference to “value” includes a plurality of such values and reference to “polygon” includes reference to one or more polygons and equivalents thereof known to those skilled in the art, and so forth.

Unless defined otherwise, all technical and scientific terms used herein have the same meaning as commonly understood to one of ordinary skill in the art to which this disclosure belongs.

The term “subject” or “patient” are used interchangeably and encompass a cell, tissue, organism, human or non-human, mammal or non-mammal, male or female, whether in vivo, ex vivo, or in vitro.

The terms “marker,” “markers,” “biomarker,” and “biomarkers” are used interchangeably and encompass, without limitation, lipids, lipoproteins, proteins, cytokines, chemokines, growth factors, peptides, nucleic acids, genes, and oligonucleotides, together with their related complexes, metabolites, mutations, variants, polymorphisms, modifications, fragments, subunits, degradation products, elements, and other analytes or sample—derived measures. A marker can also include mutated proteins, mutated nucleic acids, structural variants including copy number variations, inversions, and/or transcript variants, in circumstances in which such mutations or structural variants are useful for developing a model (e.g., a machine learning model or a cellular disease model), or are useful in predictive models developed using related markers (e.g., non—mutated versions of the proteins or nucleic acids, alternative transcripts, etc.).

The term “sample” or “test sample” can include a single cell or multiple cells or fragments of cells or an aliquot of body fluid, such as a urine or blood sample, taken from a subject, by means including venipuncture, excretion, biopsy, needle aspirate, lavage sample, scraping, surgical incision, or intervention or other means known in the art.

The phrase “phenotypic assay data” includes any data that provides information about a cell phenotype, such as, e.g., cell sequencing data (e.g., RNA sequencing data, sequencing data related to epigenetics such as methylation state), protein expression data, gene expression data, image data (e.g., high—resolution microscopy data or immune his to chemistry data), cell metabolic data, cell morphology data, and cell interaction data. In various embodiments, phenotypic assay data includes functional data, such as electrophysiological functional data for cardiac cells and electroencephalogram (EEG) or electrocorticography (ECOG) for brain cells.

The term “obtaining phenotypic assay data” encompasses obtaining any of a cell, cell population, cell culture, or organoid and capturing phenotypic assay data from any of the cell, cell population, cell culture, or organoid. The phrase also encompasses receiving a set of phenotypic assay data, e.g., from a third party that has captured the phenotypic assay data from a cell, cell population, cell culture, or organoid.

The phrase “subject data” includes phenotypic assay data determined from one or more cells that are obtained from a subject. The subject data can, in some circumstances, further include clinical data (e.g., clinical history, age, lifestyle factors, etc.) of the subject. The subject data also can, in some circumstances, include genomic and gene sequence data of the subject.

The phrase “clinical phenotype” refers to any of a disease phenotype, a presence or absence of disease, disease severity, disease pathology, disease risk, disease progression, or a likelihood of a clinical phenotype in response to a therapeutic treatment. In various embodiments, clinical phenotypes include disease—relevant clinical phenotypes that can be observed through clinical methods such as through magnetic resonance imaging. In various embodiments, clinical phenotypes include endophenotypes, which are characteristics of a disease that are not directly observable. Examples of measurements or surrogate datapoints for endophenotypes include a blood test for HbA1C levels and/or brain volume for neurological diseases. A clinical phenotype can, in some embodiments, be represented as a binary value (e.g., 0 and 1 indicating the presence or absence of disease). In some embodiments, a clinical phenotype can be represented as a continuous value (e.g., a continuous value that represents a risk associated with the disease).

The phrase “machine learning implemented method” or “ML implemented method” refers to the implementation of a machine learning algorithm, such as, e.g., any of linear regression, logistic regression, decision tree, support vector machine classification, Naïve Bayes classification, K-nearest neighbor classification, random forest, deep learning, gradient boosting, generative adversarial networking learning, reinforcement learning, Bayesian optimization, matrix factorization, and dimensionality reduction techniques such as manifold learning, principal component analysis, factor analysis, autoencoder regularization, and independent component analysis, or a combination thereof.

The phrase “cellular disease model” generally refers to a model that can be implemented for conducting experiments in a dish. Generally, a cellular disease model is a machine—learning enabled cellular disease model. For example, when deployed to perform a screen, the cellular disease model produces predictions outputted by a trained machine learning model (e.g., uses the predictions to guide the selection of an intervention). In various embodiments, the cellular disease model is a hybrid model that involves both an in vitro cellular assay component and in silico component. For example, the in vitro cellular assay component can involve testing an intervention against in vitro cells and measuring the phenotypic outputs, and the in silico component can involve interpreting the phenotypic outputs of the in vitro cells.

The phrase “therapeutic” refers to any treatment that can modify the progression or development of a disease. A therapeutic can be a small molecule drug, a biologic, an immunotherapy, a genetic therapy, or a combination thereof.

The phrase “pharmaceutical composition” refers to a mixture containing a specified amount of a therapeutic, e.g., a therapeutically effective amount, of a therapeutic compound in a pharmaceutically acceptable carrier to be administered to a mammal, e.g., a human, in order to treat a disease.

The phrase “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio.

Systems biology, the modeling and study of complex biological systems by dynamical models, seeks to understand mechanisms of individual parts by studying the whole system. These systems are usually modeled by Ordinary Differential Equations (ODEs) that model the biology of proteins binding to one another or reactions occurring within a cell. Studying the response of the system can be used to gain an understanding of latent processes underway when a cell responds to perturbations in its environments. Understanding dynamical systems of cells and how they respond to perturbations is important in drug design, where misunderstanding can lead to reduced drug efficacy and increased off-target effects. However, dynamical systems constrained by physics and experimental limitations, such as measuring a single time point after perturbing a system using flow cytometry, can lead to polynomials with non-closed form steady-state solutions that do not admit an explicit likelihood function. For example

Biological cellular systems exhibit super exponential scaling in the number of biological states achieved arising from different combinations and sequences of cell regulators, such as messenger proteins and transcription factors. This complexity impedes the understanding of diseases and development of therapeutics. The combinatorial complexity of biology, defined by the vast number of models and their parameters that describe biological systems was focused on. This combinatorial problem in biology is exemplified by promiscuous signaling, which is the phenomenon of multiple protein ligands in a pathway being able to bind to multiple receptors in a competitive manner. The Bone Morphogenetic Protein (BMP) pathway exemplifies this type of signaling with multiple protein ligands, and type I and II receptors present in the pathway, each combining with one another at different rates to form a complex of ligand, type I, and type II receptor to phosphorylate SMAD 1/5/8 to send a downstream gene expression signal. The steady state solution for a model of Bone Morphogenetic Protein (BMP) ligands binding to BMP receptors and then sending a downstream gene expression signal can be solved by least squares regression or convex optimization. While these methods provide a solution, they do not admit an explicit likelihood function that can be used directly with methods such as Markov Chain Monte Carlo (MCMC) to determine model parameters and their uncertainty. In this case, the model of BMP binding has an implicit likelihood function, which is an unknown or intractable likelihood of the data, and also known as a generative model. This is a common scenario in biology, where certain systems can be simulated but do not have an explicit likelihood function, such as systems of stochastic biological functions and metabolic pathways. The BMP pathway can be mathematically be modeled in various manners using mass action kinetics (Antebi et al., 2017) and previous work demonstrated how to optimally infer BMP models' parameters using Likelihood Free Inference (LFI), also known as Simulation Based Inference (SBI), using the SBIDOEMAN algorithm (Zaballa & Hui, 2021). However, since multiple models have been proposed for the BMP pathway (Antebi et al., 2017; Su et al., 2022), there remains ambiguity in determining which model best describes observed experimental data.

Traditional approaches to determining the parameters of a model with an implicit likelihood used Approximate Bayesian Computation (ABC) techniques, akin to guessing parameters a simulator may need to return the observed data and accepting those parameters that fall within a user-specified distance. However, this technique is slow and also typically dependent on user-defined summary statistics of the observed data, Xₒ.

Recent likelihood free inference (LFI) methods based on neural networks that estimate the density, or probability distribution, of each unknown parameter, θ, have shown to improve performance over classic ABC methods. LFI methods, also known as simulation-based inference (SBI), were recently benchmarked on various tasks and settings, and demonstrated reliably more efficient and effective in estimating parameters than ABC methods across a range of tasks.

Determining the parameters that may describe the biological system given experimental designs, p(θ|Xₒ), is important, but it is also important to design experiments to arrive at an accurate parameterization with the least number of experiments. Recent work has applied optimal experimental design to perturbation experiments to study hematopoietic stem cell (HSCs) systems and chemical design and synthesis, but there lack methods applied to perturbation-response biological settings, where the goal is understanding dynamical biological systems, such as dosing cells in microwell plates and measuring their response after an incubation period. Using uncertainty estimates, or entropy, and information-based objective functions, optimal experiments can be designed to determine parameters of dynamical systems by LFI given a model of the dynamical system, its parameter priors, and observed data.

In a certain embodiment, the methods of the disclosure provide one or more steps (e.g., machine learning steps) that utilize an algorithm for implicit biological systems that: (i) determines the parameters and their uncertainty using LFI; (ii) uses uncertainty information to design new experiments; and/or (iii) performs better than controls when benchmarked on two implicit models of the BMP signaling pathway. In a certain embodiment, various steps of a method of the disclosure are implemented using a graphic processing unit (GPU) and/or a Tensor processing unit (TPU) of a computer or server. For example, a GPU/TPU can be employed to create a machine-generated biological model. Moreover, a GPU/TPU can advantageously be employed to carry machine learning based steps. For example, a GPU/TPU can be employed in a machine learning step to infer models' parameters and models' probabilities.

Accurate parameterizations of biological systems is an ongoing area of research that has resulted in methods such as graph-based models enclosed in an activation function to parameterize models of systems biology. While previous methods may be effective at parameterizing a set of known biological connections and predicting responses to perturbation, these methods lack an uncertainty estimate that can be used to determine experiments that maximize the mutual information between prior model parameters and predictive posteriors given proposed experimental designs. Previous work has applied ABC methods to systems biology; the current disclosure innovatively extends LFI methods in systems biology by simultaneously harnessing entropy for optimal experimental designs.

As shown in the studies presented herein, it was found that the SBIDOEMAN algorithm was capable of elucidating both estimate parameters of a biophysical model with an intractable likelihood and design optimal experiments to gain more information than using a sub-optimal search strategy. The SBIDOEMAN algorithm was compared to random search as a baseline, and equidistant dosing, which is common when evaluating Hill Functions of titration curves during drug screening. The methods of the disclosure demonstrated an improvement in the rate and accuracy of parameterizing implicit biological functions over an equidistant method. This improvement is important whenever samples are scarce, such as assessing drug combinations on cancer biopsies.

The studies presented herein indicate the effectiveness of methods using the SBIDOEMAN algorithm with experimental data. The methods of the disclosure are ideally suited for experiments where multiple models are candidates to represent the true underlying biology, such as whether homodimeric and heterodimeric BMP ligands operate by different models, and potentially reduce the computational burden and increase the utility of normalizing flows for experimental design and model selection in systems biology.

The SBIDOEMAN algorithm is based on the problem that biological systems can be modeled but their parameterizations cannot be determined. Knowing the parameters is important for being able to predict how biological systems will respond to perturbations to the environment (drugs). Conventionally, one may use least squares regression to “fit” a model from observed data. However, this method lacks a measure of uncertainty and is useless besides having a single, possibly bad, fit for the data. The alternative is to use a Bayesian method to determine a distribution of parameters given the observed data. While this seems like a good solution, Bayesian methods typically rely on tractable likelihood functions, or analytical solutions to math models under study. For some biological models, there is no known analytical solution but the response can be simulated using convex optimization. By using these simulations in a process known as Likelihood-free inference (LFI), or Simulation Based Inference (SBI), the parameter distributions can be identified. With these parameter distributions, better experiments can then be designed to arrive at a more accurate model of the underlying biology.

In view thereof, the SBIDOEMAN algorithm was modified to determine the marginal probability of a model, which is the probability that a model is correct. By using this determined probability in a Bayesian framework, better experiments can be designed. The modified SBIDOEMAN algorithm is capable of determining which biological model is correct. Once known, drugs may be tested in biological disease models in a selective fashion, targeting known combinations of proteins associated with a disease. This is an improvement over traditional methods as the off-target effects can be minimized while maximizing on-target effects. Additionally, if designing a drug or biologic from scratch, this method can optimize which drug or biologic to use, or, said differently, predict which drug or biologic might have the best on and off-target effects.

Accordingly, further provided herein are methods utilizing a modified SBIDOEMAN algorithm to approximate a model's marginal probability, p( [symbol] |x₀,θ), within Bayesian Model Averaging (BMA) to select a correct model from a set of models proposed. This algorithm, termed SBIDOEMAN BMA, uses the models' prior distributions of parameters, p(θ), to design optimal experiments using a mutual information approximation I(θ,x; d) between model parameters and data, then determines the posterior distribution of parameters given observed data, p(θ|x₀), by LFI, and finally approximates a marginal likelihood of a biological model given observed data points, p( [symbol] |x₀,θ). This marginal probability is used as a probability measure of a given model, [symbol] , and can be used in BMA to determine the next experiment to evaluate and a weighting of possible models.

Previous work for optimal experimental designs in biological systems studied graphical models describing gene regulatory networks, modeled using Bayesian graphs, and M-estimators applied to Gaussian Markov Random fields, both of which have closed-form information measures. By contrast, the systems disclosed herein are geared to the LFI setting where likelihoods and closed-form information measures are not tractable. Regarding model selection, trained classifiers have been proposed to classify whether data can fit a proposed model or not. While useful in model selection, this system does not provide a posterior distribution of models' parameters or design optimal experiments. The methods of the disclosure, however, provide can be utilized for evaluating models by their likelihood function, compare models, and design experiments towards the most promising model. Additionally, the methods of the disclosure can be used with biological high throughput screening assays.

In a particular embodiment, the disclosure provides a means to determine the marginal probability of a model given observed data using the methods of the disclosure. In a further embodiment, the disclosure also provides a means for BMA to be applied to optimized experimental designs to design experiments for a given model using the methods of the disclosure. In particular, the disclosure provides methods that utilize a machine learning algorithm (i.e., SBIDOEMAN and SBIDOEMAN BMA) to design and evaluate experiments in biological models that is compatible with HTS of biological systems. It the studies presented herein, the robustness and performance of SBIDOEMAN BMA was demonstrated. More specifically, the SBIDOEMAN BMA was found to accurately model the BMP pathway over competing methods, including a standard heuristic in biological systems. By analyzing an ensemble of models, SBIDOEMAN BMA can predict optimal designs and more efficiently provide an evaluation of posterior analyses. In the process of comparing SBIDOEMAN BMA, it was shown herein how to estimate a model's marginal probability using normalizing flows in the methods disclosed herein. It was further shown with the methods of the disclosure that averaging the mutual information estimate between models resulted in designs that outperform competing methods in improving the quality of experiments.

As shown in the studies presented herein, methods of disclosure using the SBIDOEMAN BMA have been validated in two types of simple models, one-step and twostep models, of the BMP pathway, each with two and three parameters, respectively. It is expected that methods using the SBIDOEMAN BMA algorithm will scale to larger models and minimize noise and batch effects in experimental systems. While the averaging of the mutual information among models was used in methods disclosed herein to design optimal experiments, it is expected that each model's mutual information can also be weighted by its respective marginal probability in the methods of the disclosure, leading to improved designs for the model with more evidence. Additionally, while a simple ensemble method was used to evaluate the performance of iid models using the methods disclosed herein, allowing for the measurement of uncertainty in models' predictions, Mixtures of Experts (MoEs) can also be used with the methods of the disclosure to improve training and can be combined with ensembling methods to perform uncertainty quantification. These methods could both improve performance and uncertainty quantification in optimal designs for biological models.

In another embodiment the disclosure also provides a method that utilizes computer-implemented models and data from experiments in machine learning models to identify and/or rank small molecule drug(s) and/or biologic(s) or perturbagen(s) that modulate a targeted cellular biological process to a statistically significant degree. In a further embodiment the method is a machine learning enabled method. For such a method the method can utilize observable data obtained from in silico experiments with ‘simulated cells’ or observable data obtained use ‘wet bench’ biological experiments with actual cells or microorganisms. Regarding the former, examples of in silico experiments can be found in the Examples section presented below. Examples of microorganisms that can be used in the method, include bacteria and fungus.

A method disclosed herein that utilizes computer-implemented models and data from experiments in machine learning models to identify and/or rank small molecule drug(s) and/or biologic(s) or perturbagen(s) that modulate a targeted cellular biological process to a statistically significant degree, comprises an active method step of: obtaining cells from a subject or generating recombinant cells that elicit a measurable or trackable cellular functional response to small molecule drug(s) and/or biologic(s) on a targeted biological process. For this step, any type of cells may be used in the methods disclosed herein. For example, the cells may be obtained from an animal subject including, but not limited to, a mammal, invertebrate, reptile, bird, fish, and amphibians. In a particular embodiment the cells are obtained from a mammalian subject. In a further embodiment, the cells are obtained from a human patient.

For purposes of this disclosure, any method known in the art for obtaining a cell or population of cells from an animal subject may be used in the methods described herein, including common cell separation and/or isolation techniques. Cells can be obtained from a sample taken from an animal subject. Examples of such samples, include, but are not limited to, blood samples, bone marrow samples, tissue samples, urine samples, saliva samples, bile samples, plasma samples, stool samples, synovial fluid samples, cerebral spinal fluid samples, and vaginal samples. Alternatively, the cells can be obtained as cell lines purchased from any number of vendors including, ATCC, Sigma-Aldrich, Fisher Scientific, Thermo Fisher, Charles River, etc.

In a further embodiment, the cells can be recombinantly modified to express transgenes (e.g., reporter genes), knockout endogenous genes, and/or over- or under-express certain endogenous genes. In a particular embodiment, the cells have been recombinantly modified to express a reporter transgene that generates a detectable or measurable marker (e.g., fluorescence, chemiluminescence, biofluorescence, chromogenic change, etc.) that is used to track cellular functional response to small molecule drug(s) and/or biologic(s) on a targeted biological process. Further, the detectable or measurable marker can be tracked or quantified directly (e.g., by measuring light intensity) or indirectly (e.g., by adding a substrate that is acted on by an enzyme to produce chemiluminescence or chromogenic change). Cells can be recombinantly modified using any number of techniques known in the art, including gene editing systems, recombinant mutagenesis, homologous recombination, transduction-based methods, and transfection with plasmids. Additionally, or alternatively, the functional response to small molecule drug(s) and/or biologic(s) on a targeted biological process can be tracked in cells by using gene expression assays (e.g., microarrays, beadchips, genechips, etc.), sequencing techniques (e.g., RNA-Seq, transcriptome analysis) and PCR techniques (e.g., qRT-PCR).

In a particular embodiment, the cells are selected to study a targeted biological process, including but not limited to, a biological pathway associated with growth, metabolism, or interactions and communications between cells. In further embodiments, the cells that are obtained are cells that are associated with a disease or disorder. Examples of disease or disorder include, but are not limited to, an infectious disease, a deficiency disease, a genetic hereditary disease, a non-genetic hereditary disease, a physiological disease, an idiopathic disease, and a neoplastic disease. In a certain embodiment, the cells selected are associated with cancer, or cancer cells.

With regards to small molecule drugs, the drugs may be known drugs and/or novel drugs. Similarly, with regards to the biologics, the biologics may be known biologics and/or novel biologics. The biologics may be protein-based biologics. Protein-based biologics includes peptides, fragments of proteins, full proteins, or complexes of proteins.

A method disclosed herein that utilizes computer-implemented models and data from experiments in machine learning models to identify and/or rank small molecule drug(s) and/or biologic(s) that modulate a targeted cellular biological process to a statistically significant degree, comprises an active method step of: training a first machine learning model with a plurality of computer-implemented models that model the targeted biological process using user defined parameters, and which define prior probabilities in the models' parameters and models' marginal likelihood. The examples of training such a machine learning model can be found in the Examples section presented below. In a particular embodiment, the parameters of the plurality of computer-implemented models have user defined prior probabilities and marginal likelihoods. The computer-implemented models may be mathematical models, models that predict protein structures when complexed with small molecule drugs and/or biologics, or some combination thereof. Examples of models that predict protein structures when complexed with small molecule drugs and/or biologics include AlphaFold2, Rosetta, RoseTTAFold, CASP14, OmegaFold, ESM Metagenomic Atlas, and AlphaFold. In a particular embodiment, computer-implemented models comprise models that predict protein structures when complexed with small molecule drugs and/or biologics.

A method disclosed herein that utilizes computer-implemented models and data from experiments in machine learning models to identify and/or rank small molecule drug(s) and/or biologic(s) or perturbagen(s) that modulate a targeted cellular biological process to a statistically significant degree, comprises an active method step of: training a second machine learning model to estimate the mutual information between observed data and computer-implemented models' parameters, to design experiments to optimally perturb the modeled biological process with the small molecule(s) and/or biologic(s). Mutual information (MI) is a ubiquitous measure of dependency between a pair of random variables and is one of the corner stones of information theory. Experiments are designed to test small molecule drug(s) and/or biologic(s) or perturbagen(s) that are identified as being most probable to modulate a targeted cellular biological process based upon the output of the machine learning model.

A method disclosed herein that utilizes computer-implemented models and data from experiments in machine learning models to identify and/or rank small molecule drug(s) and/or biologic(s) or perturbagen(s) that modulate a targeted cellular biological process to a statistically significant degree, comprises an active method step of: performing biological experiments with the cells with small molecule drug(s) and/or biologic(s) or perturbagen(s) identified using a machine learning model to generate measurable or observable cellular functional response data, the biological experiments being designed from the plurality of computer-implemented models' prior probabilities and binding affinity of the small molecule drug(s) and/or biologic(s) or perturbagen(s) to a biological component of the targeted biological process. Generally, the biological experiments are cell-based screening assay were various concentrations or dilutions of small molecule drug(s) and/or biologic(s) or perturbagens identified by the machine learning model are added to wells of plates or dishes which contain the cells. Such addition of small molecule drug(s) and/or biologic(s) or perturbagens can be manually added to the wells or dispensed to the cells using automation equipment. With regards to the latter, the automation equipment can be part of a high throughput system. The high throughput system can further comprise equipment to measure the observable function response data, such as reader or detector for fluorescent light production. The high throughput system can further comprise equipment like heater and incubators to maintain the treated cells at a desired temperature.

A method disclosed herein that utilizes computer-implemented models and data from experiments in machine learning models to identify and/or rank small molecule drug(s) and/or biologic(s) or perturbagen(s) that modulate a targeted cellular biological process to a statistically significant degree, comprises an active method step of: retraining a machine learning model using the measured or observed cellular functional response data to update: (i) the binding affinities of the targeted biological pathway, (ii) the small molecule drug(s) and/or biologic(s) binding affinity to the biological component, and (iii) to indicate which model of the plurality of computer-implemented models most accurately models the targeted biological process; and performing one or more iterations of the machine learning model until small molecule drug(s) and/or biologic(s) are identified that perturb the targeted biological process until a Z-factor from 0.5 to 1.0 is determined. Z-factor or Z′-factor as used herein refers to a statistical data quality indicator for a bioassay, particularly that used in the field of high throughput screening (HTS). [symbol] Z-factor of 1, ideal. This value is approached when you have a huge dynamic range with tiny standard deviations. In this situation, the separation band is almost as long as the dynamic range. Z-factors can never be greater than 1.0. A Z-factor between 0.5 and 1.0 is an excellent assay. A Z-factor between 0 and 0.5 is marginal. A Z-factor less than 0 means that the signal from the positive and negative controls could overlap, making the assay not very useful or screening purposes.

In a particular embodiment, the methods of the disclosure can identify and/or rank small molecule drug(s) and/or biologic(s) or perturbagen(s) that modulate a targeted cellular biological process to a statistically significant degree. The small molecule drug(s), biologic(s) or perturbagen(s) could be known chemical entities or novel chemical entities. With regards to former, the methods of disclosure can identify known chemical entities that can be used for a new therapeutic purpose, be combined with other chemical entities to have an improved therapeutic effect, and/or be used at doses that are not normally administered. The methods of the disclosure can also be used to identify new chemical entities based upon the machine learning modeling data and permutations made thereof.

For the methods disclosed herein any of the steps that require computation (e.g., machine learning steps), these steps can be performed using the CPU and/or GPU of a computer or server or performed using an AI accelerator of a server. In a particular embodiment, the machine learning steps are carried out using a GPU of a computer. In another embodiment, the machine learning steps are carried out using an AI accelerator from a cloud-based server or web service.

### EXAMPLES

Software and Data.

The hydra configuration manager was used to track hyperparameters and seeds of experiments, according to the methods taught in Yadan (Github 2019). To perform SBI, the SBI software library according to Tejero-Cantero et al. (Journal of Open Source Software, 5(52):2505 2020)) were used. The model marginal probability calculation was performed using JAX and Distrax libraries according to Bradbury et al. (Github 2018) and Babuschkin et al. (Github, 2020).

Normalizing Flows.

Normalizing flows are a class of invertible and differentiable neural networks that describe a series of monotonic functions that can either minimize the divergence of the pushforward from a base distribution, pu(u), which is typically a Gaussian distribution, to the data px(x), or vice versa via a pullback. Formally, the change of variable formula and a composition of monotoic diffeomorphic functions, ƒ_{Ø}, which can be neural networks parameterized by Ø, to transform data from a base distribution, pu(u), to the data distribution, px(x) were used according to EQ. 1:

$$p x ( x ) = {p}_{u} ( {f}_{∅}^{- 1} ( x ) ) \vert det \frac{\partial {f}_{∅}^{- 1}}{\partial x} \vert \qquad ( 1 )$$

In parallel to recent innovations normalizing flow architectures, much work has focused on algorithms for sequential posterior estimation by estimating the posterior, likelihood, and ratios of posteriors to priors to estimate the posterior p(θ|xₒ) of a model of interest given observed data xₒ. SBI methods are used extensively in fields where functions can be simulated but not evaluated, such as particle physics. The SBI method used in this paper is known as Sequential Neural Posterior Estimation (SNPE), which uses a neural network to directly estimate the posterior distribution. SNPE aims to estimate the posterior directly, q̅_{x,Ø}, by EQ. 2:

$${\overline{q}}_{x , ∅} = {q}_{F ( x , ∅ )} ( θ ) \frac{\tilde{p} ( θ )}{p ( θ )} \frac{1}{Z ( x , ∅ )} \qquad ( 2 )$$

where q_{F(x,Ø)}(θ) is a normalizing flow that estimates the posterior p(θ|x), Z(x,Ø) is a normalization constant, and \tilde{p}(θ)/p(θ) is a user-defined importance weighting factor.

Design of Experiments (DOE) for Implicit Models.

While much recent research has focused on developing novel normalizing flow and SBI methods, DOE for models with implicit likelihoods has only recently seen increased attention, with a focus on evaluating different score functions of estimates of the mutual information's lower and upper bounds between a model's priors and predictive posterior. Commonly, most methods start by finding the optimal experimental design, d* that maximizes a utility function, U(d), describing the change in entropy of model parameters before and after an experiment with design d is conducted. This optimization problem is described as EQ. 3:

$${d}^{*} = {argmax}_{d ∈ D} U ( d ) \qquad ( 3 )$$

where D represents the space of feasible designs. The utility function can then be formulated as the mutual information, I(θ,y|d) between θ and y given a certain design d of EQ. 4:

$$U ( d ) = I ( θ , y \vert d ) = {𝔼}_{p ( θ ) p ( y \vert θ , d )} [ log \frac{p ( y \vert θ , d )}{p ( y \vert d )} ] \qquad ( 4 )$$

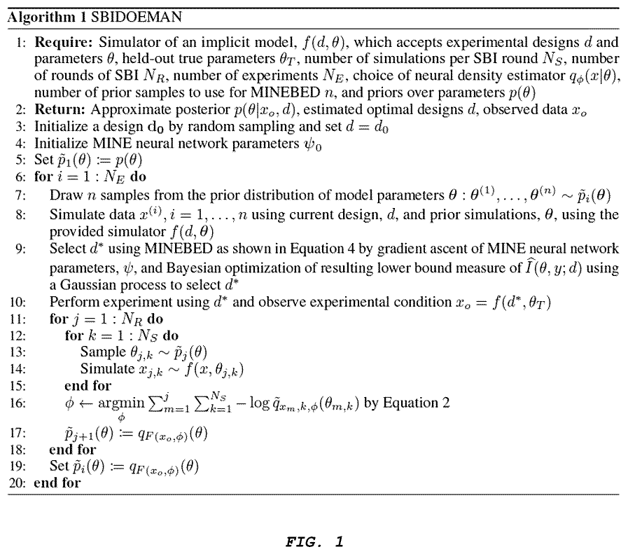

which results in the expected information gain given a certain experiment, d. Various upper and lower bound of the mutual information have been proposed. An estimate of the lower bound of the mutual information using the Donsker-Varadhan lower bound calculated by a Mutual Information Neural Estimation (MINE) network was used. This lower bound is then used as the objective function of a Gaussian process within a Bayesian Optimization routine. Altogether, these parts constitute the Simulation-Based Inference Design Of Experiment for biological Mechanistic Acyclic Networks (SBIDOEMAN) algorithm (see FIG. 1).

The SBIDOEMAN Algorithm & Choice of Hyperparameters.

The SBIDOEMAN algorithm is described using a simulator of the BMP model as a surrogate for collecting experimental data. When experimentally validating the algorithm, the experimental collection process needs to be replaced by an iterative experimental process. Potentially confusing nomenclature for the SBIDOEMAN algorithm is the difference between the number of SBI rounds, N_{S}, which is the number of rounds of posterior refinement in the SBI module, and the number of experimental rounds, N_{E}, which is the total number of experiments to perform.

When implementing SBIDOEMAN in code, there are multiple opportunities to reuse samples from different sections of the code in order to amortize sampling, but are omitted here for brevity. The hyperparameters that were chosen were NS=500, NR=2, and a NSF architecture with 150 hidden features (neurons), 10 transforms, and 20 bins. Additionally, a constrained optimization of this algorithm can be realized based on the finite resource for the number of designs, d, for DOE of implicit models include. Then, NE will be the result of the constrained optimization problem.

Physical Models of the BMP Pathway.

The BMP signaling pathway can be described by mass action kinetics of proteins binding to one another and conservation laws to describe the process of a downstream genetic expression signal reaching a steady-state based on receptors available and ligands in a cell's environment. Varying degrees of model complexity can be formulated and used to describe observed biological data. The twostep model of BMP signaling was originally proposed by Antebi et al. This system is described as n_{L} ligands, Lⱼ, binding to one of n_{A} Type Aᵢ receptor to form a heterodimeric complex, Dᵢⱼ, which then binds to one of n_{B} type Bₖ receptors to form a trimeric complex, Tᵢⱼₖ. An assumption made was that the reactions are reversible with forward rates k_{fij}^{D} and k_{fij}^{T} for dimeric and trimeric complex formation, and kᵣᵢⱼ^{D} and kᵣᵢⱼ^{T} for the reverse reaction rates. This model's chemical equilibrium equations are expressed as EQ. 5 and EQ.6:

$${A}_{i} + {L}_{j} {{⇌}^{{K}_{{f}_{ij}}^{D}}}_{{K}_{{r}_{ij}}^{D}} {D}_{ij} \qquad ( 5 )$$

$${D}_{ij} + {B}_{k} {{⇌}_{{K}_{{r}_{ijk}}^{T}}}^{{K}_{{f}_{ij}}^{T}} {T}_{ijk} , \qquad ( 6 )$$

where there is a chemical equilibrium between the formation of a Dimeric Ligand-receptor complex and trimeric complex and its respective dimeric and type B receptor.

The twostep was followed by a simpler model by Su et al. called the onestep model, modeling only one step to form the Trimeric complex of Ligand, Type A, and Type B receptors, as presented in EQ. 7:

$${A}_{i} + {B}_{k} + {L}_{j} {{⇌}_{{K}_{{r}_{ijk}}}}^{{K}_{{f}_{ijk}}} {T}_{ijk} . \qquad ( 7 )$$

The onestep model uses one less binding affinity to model the rate of downstream signal expression than the twostep model.

Both models found each complex Tᵢⱼₖ phosphorylates an intracellular second messenger at a rate ∈ᵢⱼₖ to generate gene expression signal S, which degrades at a rate γ. This differential equation is shown as EQ. 8:

$$\frac{dS}{dt} = {\sum }_{j = 1}^{{n}_{L}} {\sum }_{i = 1}^{{n}_{A}} {\sum }_{k = 1}^{{n}_{B}} {ϵ}_{ijk} {T}_{ijk} - γ S \qquad ( 8 )$$

Both onestep and twostep models can be represented by ordinary differential equations (ODEs); however, ODEs do not reflect the experimental constraints in place when modeling the reaction of cells to ligand in a contained volumetric environment where ligands do not degrade. Considering ligands do not degrade and in vitro evaluation of cells' response to ligands is measured in a microwell plate with fixed volume, conservation laws turn the ODE into an algebraic system of equations. Under this regime, where volume of ligands is large and there are significantly more ligands than receptors, ligand concentration can be assumed to remain constant. Additionally, by assuming that production and consumption of receptors are in steady state, conservation of mass of each molecule enforces a set of algebraic equations. Letting Lⱼ⁰, Aᵢ⁰, and Bₖ⁰, represent initial values of each species, for the onestep model, the following constraints (EQ. 9, EQ. 10 and EQ. 11) were obtained:

$${L}_{j}^{0} = {L}_{j} \qquad ( 9 )$$

$${A}_{i}^{0} = {A}_{i} + {\sum }_{j = 1}^{{n}_{L}} {\sum }_{k = 1}^{{n}_{B}} {T}_{ijk} \qquad ( 10 )$$

$${B}_{k}^{0} = {B}_{k} + {\sum }_{j = 1}^{{n}_{L}} {\sum }_{i = 1}^{{n}_{A}} {T}_{ijk} \qquad ( 11 )$$

The assumption of steady-state equilibrium is made because the binding and unbinding of ligands and receptors occurs at a faster time scale than downstream gene expression. Hence, the time derivatives of any ODEs vanish and the binding affinity, Kᵢⱼₖ≡K_{fijk}/Kᵣᵢⱼₖ, and phosphorylation efficiency, ∈ᵢⱼₖ=∈_{fijk}/γ turns into the algebraic equations EQ. 12 and EQ. 13:

$${T}_{ijk} = {K}_{ijk} {L}_{j} {A}_{i} {B}_{k} \qquad ( 12 )$$

$$S = {\sum }_{j = 1}^{{n}_{L}} {\sum }_{i = 1}^{{n}_{A}} {\sum }_{k = 1}^{{n}_{B}} {e}_{ijk} {T}_{ijk} \qquad ( 13 )$$

EQ. 10 and Eq. 11 by solving for steady-state values of Aᵢ and Bₖ, respectively, and combine with EQ. 12 to arrive at a system of n_{T}=n_{L}n_{A}n_{B} quadratic equations for Tᵢⱼₖ of EQ. 14:

$${T}_{ijk} = {K}_{ijk} {L}_{j} ( {A}_{i}^{0} - {\sum }_{{j}^{′} = 1}^{{n}_{L}} {\sum }_{{k}^{′} = 1}^{{n}_{B}} {T}_{{ij}^{′} {k}^{′}} ) ( {B}_{k}^{0} - {\sum }_{{j}^{′} = 1}^{{n}_{L}} {\sum }_{{i}^{′} = 1}^{{n}_{A}} {T}_{{i}^{′} {j}^{′} {k}^{′}} ) . \qquad ( 14 )$$

The solutions for Tᵢⱼₖ can be substituted into EQ. 13 and solved by least squares regression or convex optimization. However, an explicit solution is not readily available, as solving the equation results in multiple positive, real-valued, discriminant solutions that can be distinguished in simple models by qualitative interpretation of the solutions. Thus, difficulty in determining the discriminant makes this model of BMP signaling an implicit model.

Choice of Normalizing Flow.

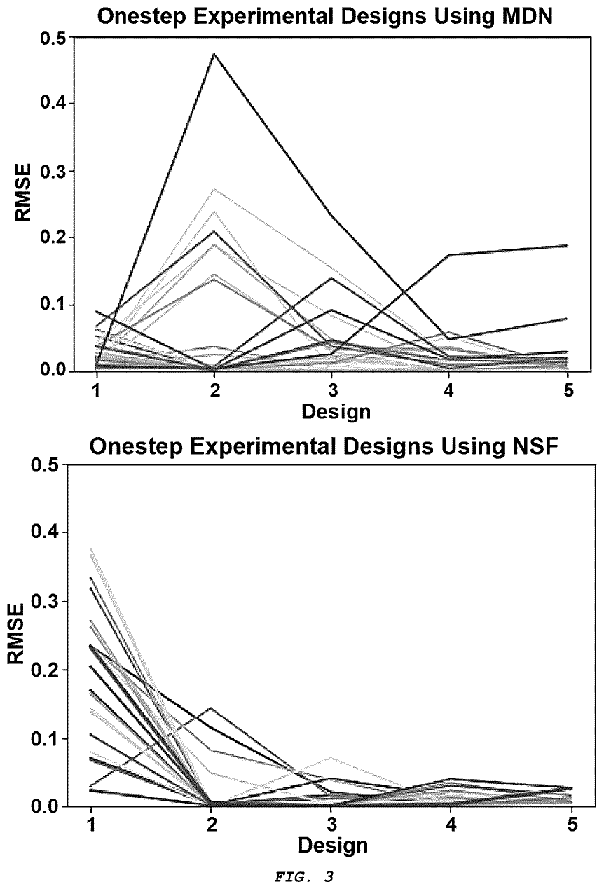

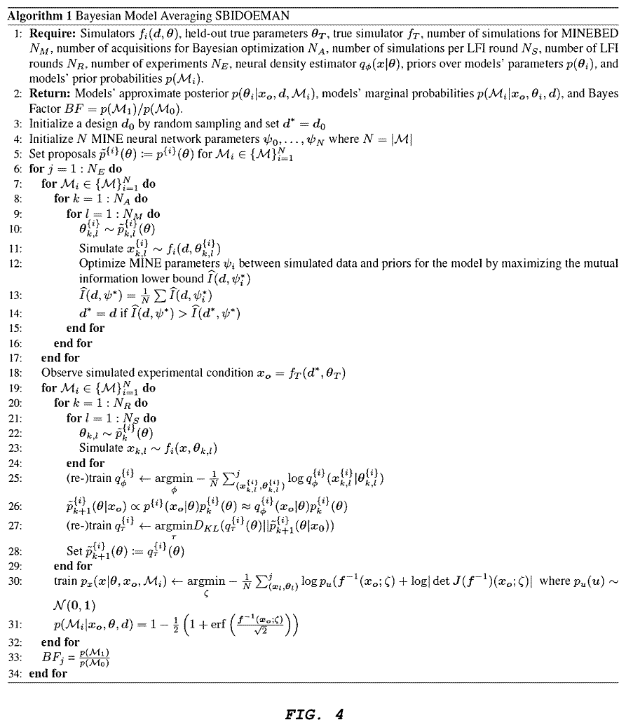

An important choice when conducting SBI is the type of normalizing flow used, where there are tradeoffs between computational complexity and accuracy. A simple neural network that was tested was the Mixture Density Network trained by Stochastic Variational Inference (SVI). This network is easy to sample but not as sensitive to non-Gaussian distributions. Another option that was considered were neural spline flows, which are flexible likelihood estimators that are relatively fast to perform inference and sampling. Using an ensemble of neural density estimators can help to evaluate the performance of the choice of normalizing flow for the task at hand. It was noticed that an improvement in the simple onestep BMP model when switching from a MDN to an NSF, as denoted by the decrease in variance of MAP RMSE over subsequent experimental design rounds and shown in FIG. 3.

Modeling the BMP Pathway.

Two mass action kinetics models have been proposed for the BMP pathway. The one-step model in EQ. 15 models type I (A) and type II (B) receptors and a ligand (L) forming a trimer complex in a single step (Su et al., 2022):

$$A + B + L {\to }^{K} T \qquad ( 15 )$$

The two-step model in EQ. 16 and EQ. 17 adds a parameter to model a ligand first binding with a type I receptor before forming a trimeric complex with a type II receptor (Antebi et al., 2017) as follows

$$A + L {\to }^{{K}_{1}} D \qquad ( 16 )$$

$$B + D {\to }^{{K}_{2}} T \qquad ( 17 )$$

Both models have a complex, T, that phosphorylates SMAD to send a downstream gene expression signal, S, with a certain efficiency, ϵ as in EQ. 18:

ϵT=S (18)

Steady-state signals can be simulated using convex optimization (Su et al., 2022).

Normalizing Flows.

Given a dataset, one may ask what is the probability of a certain data point in the dataset, px(x), of a variable x with [symbol] ^{D}RD dimensions. However, this probability density is usually intractable or unknown. Normalizing flows provide a way to answer this question by creating a transformation from a known simple distribution, pu(u), such as a Gaussian distribution, to the data distribution, px(x), by a series of nonlinear and invertible composition of functions, ƒ: [symbol] ^{D}→ [symbol] ^{D}, where ƒ is composed of N functions, ƒ=ƒ_{N} ∘ . . . ∘ƒ₁. A base distribution to target distribution can be mapped using the change-of-variables formula for random variables as EQ. 19:

px(x)=pu(u)|detJ(ƒ)(u)|⁻¹ (19)

where J(ƒ)(u) is the Jacobian matrix of ƒ evaluated at u. See Murphy (2023) for details about normalizing flows.

Likelihood Free Inference.

For models with an implicit or intractable likelihood function, p(x|θ), but whose response may be simulated, LFI methods can be used to approximate the posterior q(θ|x) or likelihood q(x|θ). This can be done by drawing N samples from the prior p(θ) and generating a dataset {(θₙ,xₙ)}ₙ₌₁^{N} by sampling θₙ˜p(θ). Each (θₙ, xₙ) is a joint sample from p(θ,x)=P(θ)p(x|θ), and can be used to train a normalizing flow to approximate the posterior q(θ|x) conditioned on an observed xₒ (Greenberg et al., 2019; Papamakarios & Murray, 2016) or approximate the likelihood q(x|θ) conditioned on θ. See Papamakarios et al. (2019) for details on applying normalizing flows to LFI.

While LFI provides a method to approximate a model's posterior or likelihood, practical considerations, such as difficulty in rejection sampling in in sequential neural posterior estimate (SNPE) (Greenberg et al., 2019) or prohibitively slow MCMC sampling for sequential neural likelihood estimate (SNLE) (Papamakarios et al., 2018), make LFI methods difficult to implement. In response to this difficulty, recent methods have developed variational methods to approximate the posterior or likelihood. These methods, referred to here as sequential neural likelihood variational inference (SNLVI), train another normalizing flow, q_{ϕ}(θ), to minimize the divergence from an estimated likelihood, ϕ*=argminϕD(q_{ϕ}(θ)∥q_{ψ}(x|θ)). SNLVI methods are used to overcome prior practical difficulties in LFI methods.

Optimal Experimental Design for Implicit Likelihood Model Selection.

Optimal experimental designs (OEDs) can be formulated as an optimization or information theoretic problem. Assuming designs are independent of model parameters, this problem is formulated as maximizing the information gain (IG), or, the difference in entropy given a proposed design, d, as EQ. 20:

IG(x,d)=H[p(θ)]−H[p(θ|x,d)] (20)

This objective function can be rewritten as a utility function, U(d), that maximizes the mutual information (MI), I(v; y|d) between a variable of interest, v, and the observed data, x, at particular design, d. The MI variable of interest, v, can be adapted to the scientific question at hand (Ryan et al., 2016). A gradient-based approach for OEDs was recently proposed for likelihood free models that provides a way to both select a model, [symbol] , by BMA and determine its parameters, p(θ| [symbol] ) with a minimum number of experiments (Kleinegesse & Gutmann, 2021). Finding designs that optimally discover a model and its parameters can be formulated as the following utility function of EQ. 21:

$$U ( d ) = {\sum }_{ℳ} \int p ( x \vert {θ}_{ℳ} , ℳ , d ) p ( {θ}_{ℳ} , ℳ ) log ( \frac{p ( {θ}_{ℳ} , ℳ \vert x , d )}{p ( {θ}_{ℳ} , ℳ )} ) dx \qquad ( 21 )$$

EQ. 21 is implemented by simply averaging each model's Mutual Information Neural Estimation (MINE) (Belghazi et al., 2018) MI estimate. The estimated MI is then used as the objective function in Bayesian Optimization using a Gaussian Process (Kleinegesse & Gutmann, 2020).

Bayesian Model Averaging and the Bayes Factor.

The weighting of model probabilities is also known as the Bayes Factor (BF), which are defined herein as BF=p( [symbol] ₁)/p( [symbol] ₀), and can be used as a form of model selection where BF>10 is strong evidence for [symbol] ₁ and BF<1/10 is strong evidence for [symbol] ₀. The BF is used for model selection as it uses marginal probabilities that prefer simpler models by the Bayesian Occam's razor effect. Although, this relies on an accurate estimate of the model's marginal probability. See Murphy (2022) for further discussion on various model selection techniques.

Approximating Model Marginal Probability.

To perform model selection, an estimate of each model's marginal probability is needed in order to calculate the BF. To do this, a normalizing flow can be used with a Gaussian base distribution pᵤ(u) that provides a probability of a model given the posterior parameter distribution and observed data, p( [symbol] |xₒ,θ,d), which is the same as marginal likelihood, p(xₒ|θ, [symbol] ,d), when assuming uniform priors over models, p( [symbol] ᵢ)=1/| [symbol] |. This flow is trained by sampling data from the simulator of [symbol] to produce x˜pₓ(x|xₒ, [symbol] ,θ) that can be used to train a reverse flow function to a base Gaussian distribution u=ƒ⁻¹(x). The following method to approximate the marginal likelihood.

Proposition 2.1.

The marginal likelihood of a model, [symbol] , given an observed data vector, x₀, and the model's parameters, θ, can be approximated as p(xₒ| [symbol] )≈1−Φ(ƒ−1(x₀)), where ƒ⁻¹ is the pullback of a trained normalizing flow from the observed data distribution, pₓ(xₒ), to a Gaussian base distribution, pᵤ(u), and Φ is cumulative distribution function of a Gaussian distribution.

Results of SBIDOEMAN in a BMP Pathway Model.

SBIDOEMAN was evaluated on how it performed on two simple models of the BMP pathway, called the onestep and twostep models, with held-out parameters representing the binding affinity and phosphorylation efficiency of physically-relevant variables in the BMP model. The SBIDOEMAN algorithm was compared with random experimental designs and log-equidistant titrations of ligands from 10⁻³ to 10³ ng/mL of BMP ligand as a design with a budget of 5 experimental designs for each condition. The same SNPE-based SBI with neural spline flow (NSF) normalizing flow was used for each experimental design policy tested. For each model, an ensemble of independent SNPE density estimators were trained with a sample size varying from 38 to 50 completed inferences given a time budget of 8 hours to complete. Using independent ensembles helped determine a distribution of reported metrics and was a valuable tool for debugging SBIDOEMAN.

The performance was compared by the root mean squared error (RMSE) discrepancy between the maximum a posteriori (MAP) point estimate of the inferred posterior distribution, p(θ|xₒ) and known true parameter values, θ_{T}. The results of SBIDOEMAN on the onestep and twostep models are shown in Table 1.

TABLE 1
Mean and standard error of RMSE of an ensemble of MAP estimate of the posterior
compared to true held-out parameter values after 5 sequential experimental evaluations
of SBIDOEMAN compared to random search and equidistant controls. Lower RMSE is
better. The number of samples vary due to rejection sampling from the posterior
surpassing the 8-hour allocated simulation budget. Results indicate that for two
models of the BMP pathway, SBIDOEMAN was able to perform an order of magnitude
better than random and equidistant search with no, or minimal, overlap of standard
errors for the onestep model, and better for the twostep model.

| | Policy | | |
|---|---|---|---|
| BMP Model Type | SBIDOEMAN | Random | Equidistant |
| Onestep | 0.004 ± 0.007 (n = 48) | 0.013 ± 0.035 (n = 38) | 0.023 ± 0.051 (n = 50) |
| Twostep | 0.149 ± 0.153 (n = 48) | 0.242 ± 0.146 (n = 40) | 0.249 ± 0.173 (n = 50) |

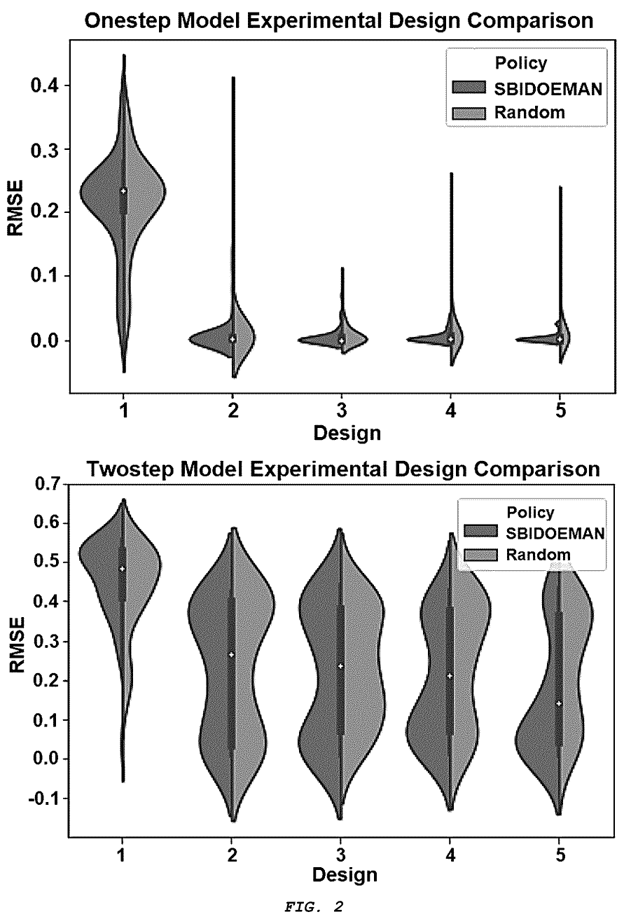

The SBIDOEMAN outperformed each control policy using a RMSE metric. To gain a better understanding of the difference in policy between SBIDOEMAN and random search, violin plots representing the posterior distribution of an ensemble of distributions representing the RMSE of the MAP estimate over the 5 designs, as shown in FIG. 2, were examined. The improvement in policy compared to the random search is clear in the simpler onestep BMP model, where random search has wider variance after the initial design, and subtly shows in the more complicated twostep BMP model in the last design.

Results of SBIDOEMAN BMA in a BMP Pathway Model.

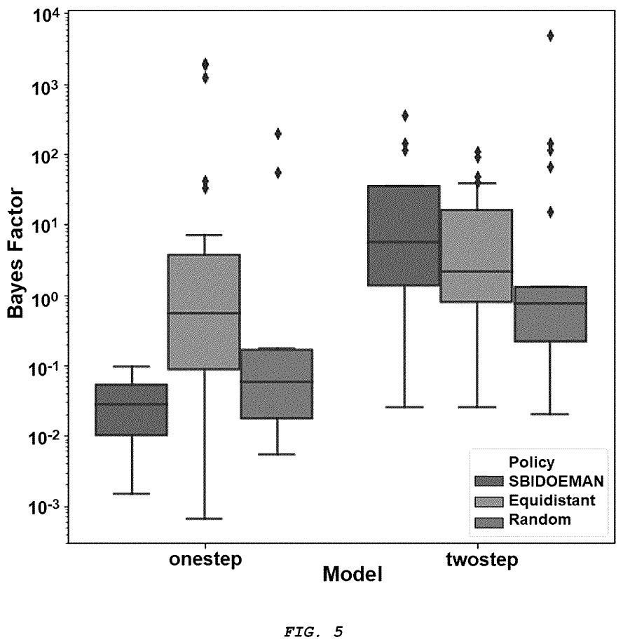

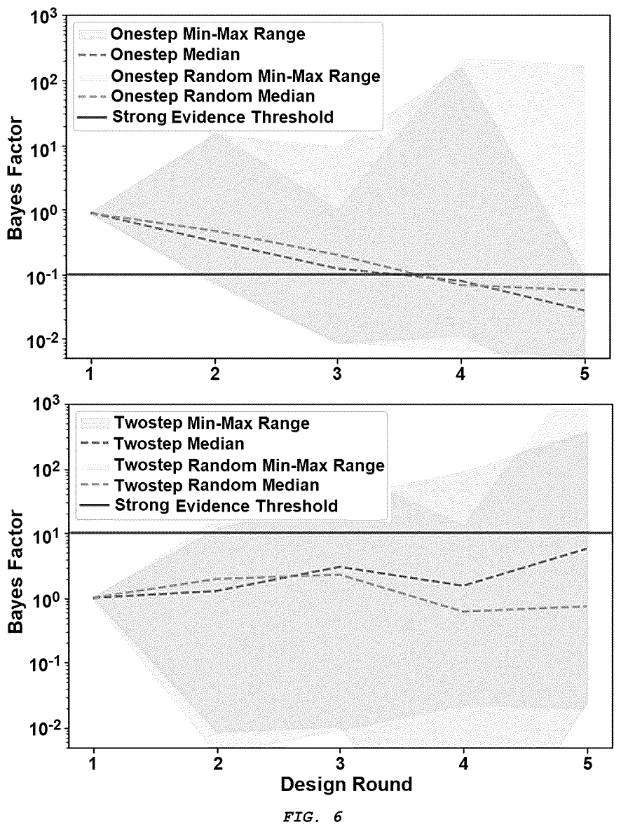

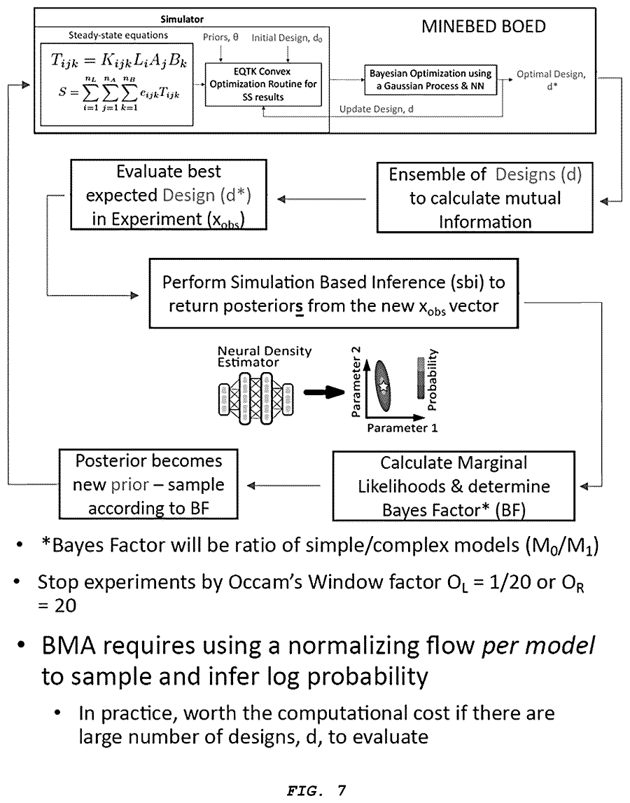

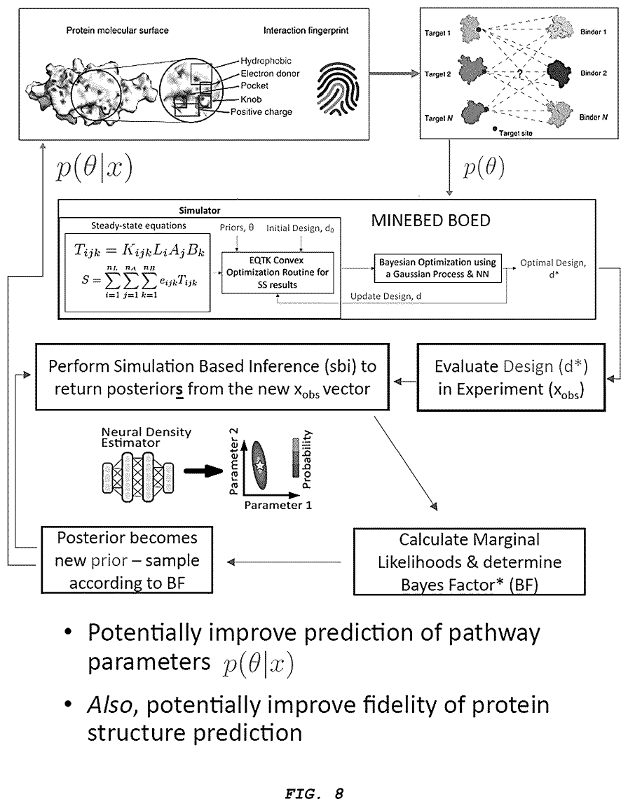

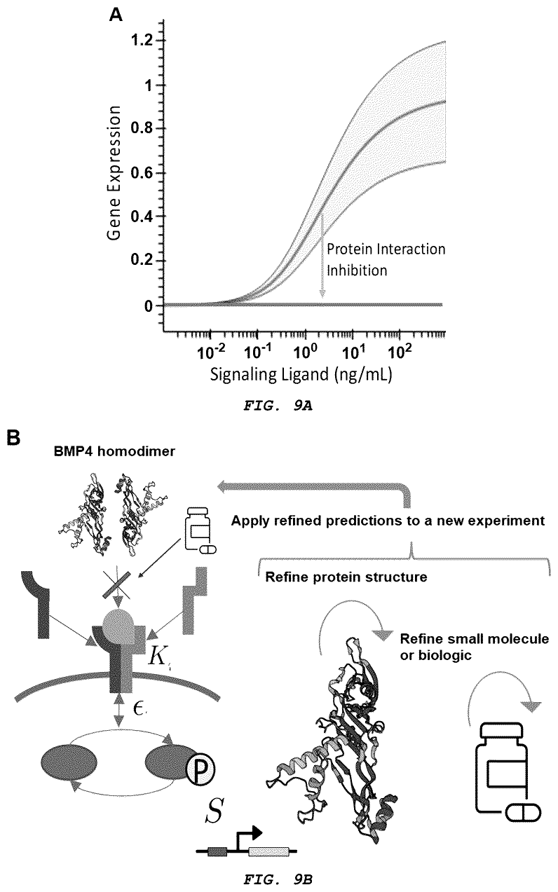

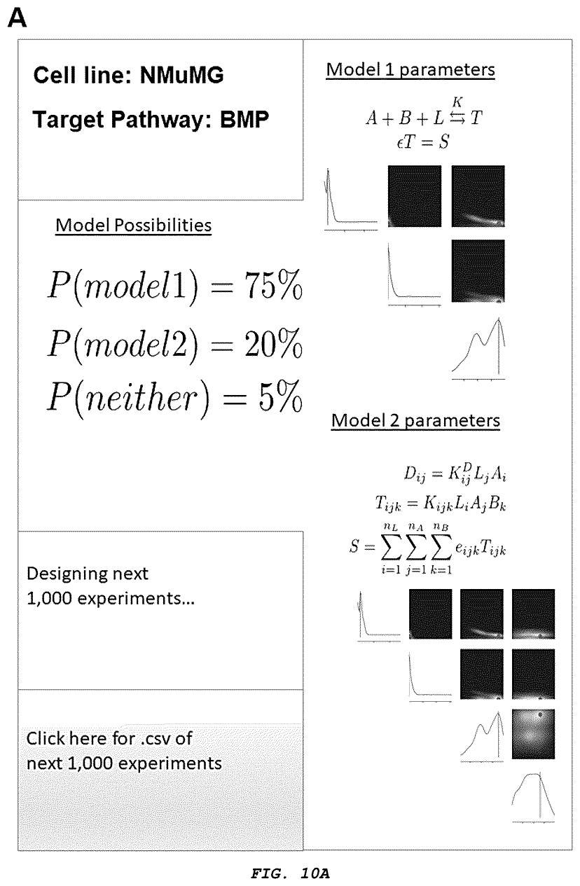

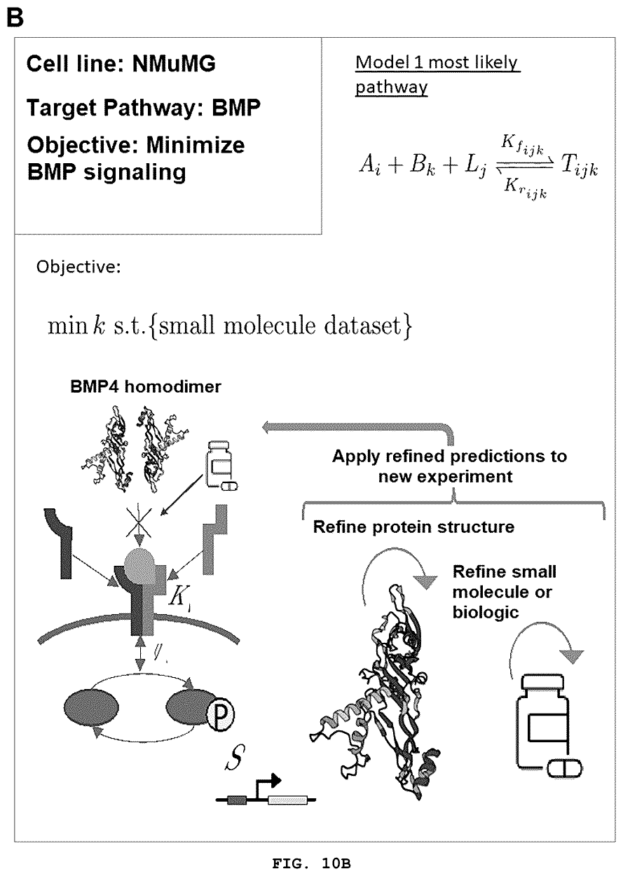

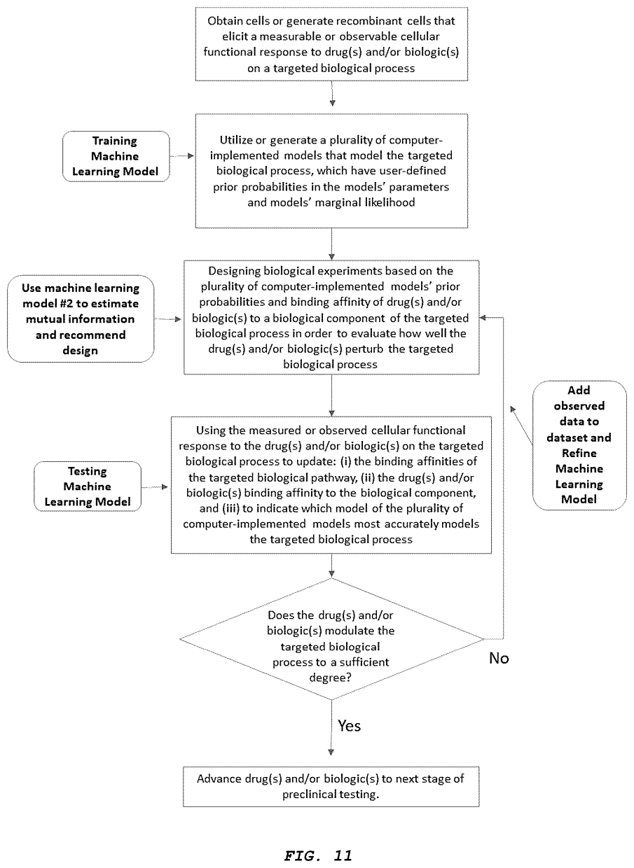

SBIDOEMAN BMA was evaluated for model selection by evaluating the BF over five rounds of experiments when the one-step BMP pathway was true and when the two-step BMP pathway was true by holding out a single set of parameters for each model, θ{1,2}T. When evaluating performance across designs, random search, as shown in FIG. 6, was compared. Final BF was compared with random and equidistant ligand titrations which is a heuristic commonly used in biology to evaluate the response of an assay. Equidistant designs are logarithmically equal spaced designs across a domain of interest. Here, this would be five equally spaced designs in concentrations from 10⁻³ to 10³ ng/mL. Results of the final design comparison are shown in FIG. 5 and Table 2.

TABLE 2
Median and interquartile range (IQR) Bayes Factor (BF) values after
5 rounds of experiments for both one-step and two-step datasets
compared to random and equidistant experimental design policies.
Lower BF is better for the series of one-step models while higher
BF is better for the two-step model. For both models, both the
median and IQR values are better than competing approaches.

| Policy | Median BF | 25% | 75% |
|---|---|---|---|
| ONE-STEP RANDOM | 0.05 | 0.02 | 0.17 |
| ONE-STEP EQUI | 0.55 | 0.09 | 3.72 |
| ONE-STEP SDM BMA | 0.03 | 0.01 | 0.05 |
| TWO-STEP RANDOM | 0.74 | 0.22 | 1.28 |
| TWO-STEP EQUI | 2.12 | 0.79 | 16.11 |
| TWO-STEP SDM BMA | 5.70 | 1.38 | 34.66 |

Examining the change in BF across designs in FIG. 6, it was found that across an ensemble of independent and identically distributed (iid) SBIDOEMAN models that the median performance outperforms random search for both the one-step and two-step models. When looking at the final BF after a budget of 5 designs, as shown in Table 2 and FIG. 6, it was found that the median performance of SBIDOEMAN BMA outperformed random and equidistant data, with SBIDOEMAN BMA interquartile range (IQR) values performing better, or almost better, than competing policy median values. While random search performed as well as SBIDOEMAN BMA in the one-step model, it performs worse in the more complex two-step model, suggesting that principled heuristics and optimal experimental design algorithms are needed for more complex models of biology.

Tissue Culture and Cell Lines.

NMuMG (NAMRU Mouse Mammary Gland cells, female) and NIH3T3 (mouse fibroblast, male) cells are acquired from ATCC (CRL-1636 and CRL-1658, respectively). E14 cells (mouse embryonic stem cells, E14Tg2a.4, male) are obtained from researchers. All cells are cultured in a humidity-controlled chamber at 37° C. with 5% CO 2. NMuMG cells were cultured in DMEM supplemented with 10% FBS (Clonetech #631367), 1 mM sodium pyruvate, 1 unit/mL penicillin, 1 ug/mL streptomycin, 2 mM L-glutamine and 1×MEM non-essential amino acids. NIH-3T3 cells are cultured in DMEM supplemented with 10% CCS (Hyclone #SH30087), 1 mM sodium pyruvate, 1 unit/mL penicillin, 1 ug/mL streptomycin and 2 mM L-glutamine. ES cells are plated on tissue culture plates pre-coated with 0.1% gelatin and cultured in a standard pluripotency-maintaining conditions using DMEM supplemented with 15% FBS (ES qualified, Gibco #16141), 1 mM sodium pyruvate, 1 unit/mL penicillin, 1 ug/ml streptomycin, 2 mM L-glutamine 1×MEM non-essential amino acids 55 mM (3-mercaptoethanol and 1000 Units/mL leukemia inhibitory factor (LIF).

Recombinant Sensor Cell Lines Construction.

Construction of the reporter cell lines is carried out via random integration of a plasmid harboring the BMP response element (BRE) in the enhancer region of a minimal CMV driving the expression of an H2B-Citrine protein fusion. ES cells are transfected using the FugeneHD reagent. NMuMG and 3T3 cells were transfected using Lipofectamine LTX. After transfection, cells are selected with 100 ug/ml hygromycin. All experiments are performed with clonal populations, generated via colony picking (ES) or limiting dilutions (NMuMG, NIH3T3). To ensure results are not dependent on the specific reporter integration site, an independent BRE-reporter cell line is generated using Piggybac integration (SBI).

BMP Response and Flow Cytometry.

Recombinant sensor cell lines are plated at 40% confluency in 96 well plates and cultured under standard conditions (above) for 12 h. Media is then replaced, and ligand(s) are added at specified concentrations. 24 h after compound addition cells are prepared for flow cytometry in the following way: Cells are washed with PBS and lifted from the plate using either 0.05 ml Accutase (ES cells) or trypsin (NMuMG and 3T3 cells) for 5 minutes at 37° C. Protease activity is quenched by re-suspending the cells in HBSS with 2.5 mg/mL Bovine Serum Albumin (BSA). Cells are then filtered with a 40 μm mesh and analyzed by flow cytometry (MACSQuant VYB, Miltenyi). All recombinant BMP ligands are acquired from R&D Systems, with the exception of BMP4, BMP10 and GDFS that are acquired from Peprotech.

Quantitative PCR (qPCR).

Total RNA is harvested from cell lysate using the RNAeasy mini kit (Qiagen) and cDNA is generated from one microgram of RNA using the iScript cDNA synthesis kit (BioRad) following the manufacturer's instructions. Primers and probes for specific genes are purchased from IDT. Reactions are performed using 1:40 dilution of the cDNA synthesis product with either IQ SYBR Green Supermix or SsoAdvanced Universal probes Supermix (BioRad). Cycling is carried out on a BioRad CFX96 thermocycler using an initial denaturing incubation of 95° C. for 3 minutes followed by 39 cycles of (95° C. for 15 seconds, followed by 60° C. for 30 seconds). Each condition is assessed with two biological repeats and each reaction was run at least in triplicate.

Time Lapse Imaging.

Fluorescent reporter cells are first mixed with an excess of non-fluorescent parental cells at a 1:9 ratio to simplify image segmentation and data extraction. Cells are then plated at 1.6·10⁴ cells/well in a 96 well plate equivalent roughly to 15-20% confluency. Cells are grown for 12 hours prior to ligand addition. Each position is imaged every hour starting from the addition of ligands until cells became confluent after about 60 h. Images are then analyzed for the number of fluorescent cells and fluorescent signal level

Protein Structure to Inform Models of Dynamics.

Integration between experimental and simulation tools have proven helpful in reasoning about complex protein structures. The advent of Alphafold 2 (AF2) demonstrated further progress in this area, such as combining cryogenic electron microscopy methods with protein structure predictions to determine the structure of the nuclear pore complex, a structure directly correlated in genetic diseases and cancers. The disclosure extends the capabilities of structural and dynamic simulation to inform experimental biology, and vice versa.

AF2 has demonstrated its ability to provide a confidence score about a complex via the predicted local-distance difference test (pLDDT), a measure of local atomic differences and derived from x-ray crystallography data. In addition to single-protein structures, AF2 can predict multimer complex formation, which is a complex formed from one or more protein structures.

This information can be used to determine the confidence in different protein multimer structures. Each dynamics model will correspond to different structures that are predicted by AF2, and each will have a confidence score. A nonoptimal method is to simply take the most confident score as the most likely complex and use that to inform dynamic models. However, the confidence score of AF2 is a point estimate, \hat{α}, rather than a distribution p(α). AF2 can also provide a distribution of scores via dropout, which is a method to approximate model uncertainty. This distribution can be included in the EIG formula and help determine which dynamical model is the correct model (see EQ. 22):

$$EIG ( ξ ) \geq {𝔼}_{p ( α ) ( m \vert α ) p ( ψ \vert m ) p ( y \vert m , ψ , ξ )} [ log \frac{{q}_{ℓ} ( y \vert m , ξ )}{{\sum }_{{m}^{′}} p ( {m}^{′} ) {q}_{ℓ} ( y \vert {m}^{′} , ξ )} ] \qquad ( 22 )$$

While this allows the optimization of a single type of experiment, it can be expanded to determine which type of experiment is most valuable. This is as simple as considering n utility functions in a set of N types of experiments and performing the n* type of experiment with the maximum utility and the maximally informative design, ξ*, as in EQ. 23:

$${n}^{*} , {ξ}^{*} = \vert \begin{matrix}argmax & argmax \\ n ∈ N \vert & ξ ∈ 𝒟\end{matrix} {\sum }_{n}^{N} {U}_{{I}_{n}} \qquad ( 23 )$$

where the optimal design is implicitly nested in the utility function. Thus, if an experimentalist must decide between acquiring more dynamical or structural data, they can simply optimize the EIG for both experiments and perform the one with maximal information.

Protein Structure and Dynamics to Inform Therapeutics.

These different sources of information can be integrated for search the better therapeutics to treat diseases. Math models of protein pathways can be used to predict downstream events based on physically relevant binding affinities, then it becomes known how changing binding affinities influences downstream events. If changing binding affinities is related to the physical structure of proteins, then drugs which interfere with specific proteins can be optimized to modulate downstream gene expression while minimizing off-target events. Targeting these types of cellular events is a subset of therapeutic development called protein-protein interaction (PPI) inhibition.

The binding affinity collected for drug screening repositories is similar to the binding affinity being inferred, with the caveat that the collected dynamics data is more faithful to actual underlying binding affinity. This is because cellular dynamics are much different than in vitro screens of protein binding affinities due to intracellular interactions, ligand-ligand binding, and potentially unknown cellular interactions that are not captured by in vitro data.

If a PPI inhibitor is being designed to alter the binding of proteins in the BMP pathway, for example, in vitro data can be included in a hierarchical Bayesian model. In this scenario, minimization of the downstream signal in the BMP pathway is preferred, S, in order to design a drug, p(γ), that influences the model parameters of a given model as, p(ψ|m), to achieve the desired downstream signal. The EIG formula can be updated as EQ. 24.

$$EIG ( ξ ) \geq {𝔼}_{p ( m ) p ( γ ) p ( ψ \vert m , γ ) p ( y \vert m , ψ , ξ )} [ log \frac{{q}_{ℓ} ( y \vert m , ξ )}{{\sum }_{{m}^{′}} p ( {m}^{′} ) {q}_{ℓ} ( y \vert {m}^{′} , ξ )} ] \qquad ( 24 )$$

A number of embodiments have been described herein. Nevertheless, it will be understood that various modifications may be made without departing from the spirit and scope of this disclosure. Accordingly, other embodiments are within the scope of the following claims.

What is claimed is:

1. A method that utilizes computer-implemented models and data from biological experiments in machine learning models to identify and/or rank small molecule drug(s) and/or biologic(s) that modulate a targeted cellular biological process to a statistically significant degree, the process comprising:
(A) obtaining cells from a subject or generating recombinant cells that elicit a measurable or trackable cellular functional response to small molecule drug(s) and/or biologic(s) on a targeted biological process;
(B) training a first machine learning model with a plurality of computer-implemented models that model the targeted biological process using user defined parameters, and which define prior probabilities in the models' parameters and models' marginal likelihood;
(C) training a second machine learning model to estimate the mutual information between observed data and computer-implemented models' parameters, to design experiments to optimally perturb the modeled biological process with the small molecule(s) and/or biologic(s);
(D) performing biological experiments with the cells from step (A) with small molecule drug(s) and/or biologic(s) identified from step (C) to generate measurable or observable cellular functional response data, the biological experiments being designed from the plurality of computer-implemented models' prior probabilities and binding affinity of the small molecule drug(s) and/or biologic(s) to a biological component of the targeted biological process;
(E) retraining the second machine learning model of step (C) using the measured or observed cellular functional response data to update: (i) the binding affinities of the targeted biological pathway, (ii) the small molecule drug(s) and/or biologic(s) binding affinity to the biological component, and (iii) to indicate which model of the plurality of computer-implemented models most accurately models the targeted biological process;
(F) repeating steps (C) to (E) until small molecule drug(s) and/or biologic(s) are identified that perturb the targeted biological process until a Z-factor of 0.5 to 1.0 is determined, wherein if a plurality of small molecule drug(s) and/or biologic(s) are identified then the method ranks the small molecule drug(s) and/or biologic(s) by their activity in perturbing the targeted biological process.

2. The method of claim 1, wherein the recombinant cells comprise a reporter gene or marker that is used to measure or track the cellular functional response to small molecule drug(s) and/or biologic(s) on a targeted biological process.

3. The method of claim 2, wherein the cellular functional response to small molecule drug(s) and/or biologic(s) on a targeted biological process can be measured or tracked using luminescence, fluorescence or chemiluminescence produced by the reporter gene or marker.

4. The method of claim 1, wherein the cellular functional response to small molecule drug(s) and/or biologic(s) on a targeted biological process can be measured or tracked based upon changes in gene expression.

5. The method of claim 4, wherein gene expression can be measured or tracked using microarrays, sequencing, immunoassays, or biochips.

6. The method of claim 4, wherein the cells obtained from a subject or the recombinant cells, are associated with a disease or disorder.

7. The method of claim 6, wherein the disease or disorder is selected from an infectious disease, a deficiency disease, a genetic hereditary disease, a non-genetic hereditary disease, a physiological disease, an idiopathic disease, and a neoplastic disease.

8. The method of claim 1, wherein one or more of the biological experiments are performed using high throughput screening with small molecule drugs and/or biologics from compound libraries.

9. The method of claim 1, wherein the biologic(s) are proteins or peptides.

10. The method of claim 1, wherein the plurality of computer-implemented models are mathematical models and/or models that predict protein structures when complexed with small molecule drugs and/or biologics.

11. The method of claim 1, wherein the targeted biological process is a targeted biological signaling pathway.

12. The method of claim 11, wherein the targeted biological signaling pathway is associated with a disease or disorder.

13. The method of claim 11, wherein the small molecule drugs and/or biologics modulate the activity of a biological component of the targeted biological signaling pathway.

14. The method of claim 11, wherein the targeted biological signaling pathway regulates growth, metabolism, or interactions and communications between cells.

15. The method of claim 1, wherein the parameters of the plurality of computer-implemented models have user defined prior probabilities and marginal likelihoods.

16. The method of claim 1, wherein the machine learning model is carried out using an AI accelerator.

17. A method that utilizes computer-implemented models and data from biological experiments in a machine learning model to identify and/or perturbagen(s) that modulate a biological pathway to a statistically significant degree, the process comprising:
(1) predicting the effect of perturbagen(s) on a biological pathway in a cellular system by using a plurality of different computer-generated models, wherein each computer-generated model provides a probable result as to the effect of perturbagen(s) on the biological pathway;
(2) providing cells or a cellular system that elicits a measurable or trackable cellular functional response to perturbagen(s);
(3) contacting the cells or cellular system with varying concentrations and/or combinations of perturbagens to modulate the activity of the biological pathway, and capturing phenotypic data resulting therefrom;
(4) training a first machine learning model with the phenotypic data to infer the uncertainty distribution of parameters of the plurality of computer-generated models, and the probable results of each computer-generated model;
(5) using the uncertainty distribution of parameters of the plurality of computer-generated models and the probability from each biological model to design additional sets of biological experiments in step (3), wherein steps (3)-(5) are repeated until perturbagen(s) are identified that perturb the biological pathway with a Z-factor from 0.5 to 1.0; and
(6) optionally, designing additional small molecule drugs and/or protein biologics based upon chemically modifying the perturbagen(s) identified in step (5).

18. The method of claim 17, wherein the plurality of different computer-implemented models are mathematical models and/or models that predict protein structures when complexed with perturbagen(s).

19. The method of claim 17, wherein the cellular functional response to perturbagen(s) on biological pathway can be measured or tracked using luminescence, fluorescence or chemiluminescence produced by a reporter gene or marker, or by measuring changes in gene expression.

20. The method of claim 17, wherein the cells or cellular system are contacted with varying concentrations and/or combinations of perturbagens using a high through screening assay.
